(19) United States
(12) Patent Application Publication
(10) Pub. No.: US 2011/0200646 A1
(43) Pub. Date: Aug. 18, 2011

(54) The TRPVL Antagonists SB-795498 For Treating Rhinitis

(76) Inventors: Charanjit Bountra, Essex (GB); Jane Christina Denyer, Hertfordshire (GB)

(21) Appl. No.: 13/062,015

(22) PCT Filed: Sep. 1, 2009
(86) PCT No.: PCT/EP2009/061236

§ 371 Date: Mar. 3, 2011

Related U.S. Application Data
(60) Provisional application No. 61/093,517, filed on Sep. 2, 2008.

(51) Int. Cl. A61K 31/4439; A61K 9/14; A61P 11/02; A61P 37/08; A61K 31/573

(52) U.S. Cl. 424/400; 514/343; 514/171

(57) ABSTRACT

The present invention relates to the use of urea compound in the treatment of rhinitis, to aqueous pharmaceutical compositions containing said compound, in particular to compositions suitable for intranasal administration.

The present invention relates to the use of a urea compound in the treatment of rhinitis, to aqueous pharmaceutical compositions containing said compound, in particular to compositions suitable for intranasal administration.

Patent application WO03/022809 discloses a series of urea compounds that have vanilloid receptor (VR1) antagonist activity. VR1 has now been renamed as the transient receptor potential vanilloid 1 (TRPV1).

It has now been found that the compound N-(2-bromophenyl)-N′-[((R)-1-(5-trifluoromethyl-2-pyridyl)pyrrolidin-3-yl)]urea, that is to say, Compound 1

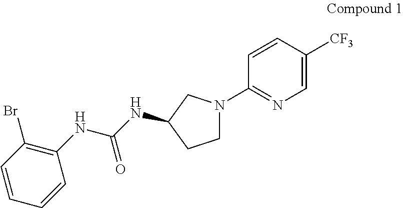

or a pharmaceutically acceptable salt thereof is useful in the treatment of rhinitis.

In one aspect of the invention, there is provided N-(2-bromophenyl)-N′-[((R)-1-(5-trifluoromethyl-2-pyridyl)pyrrolidin-3-yl)]urea or a pharmaceutically acceptable salt thereof for use in the treatment of rhinitis.

In another aspect of the invention there is provided the use of N-(2-bromophenyl)-N′-[((R)-1-(5-trifluoromethyl-2-pyridyl)pyrrolidin-3-yl)]urea or a pharmaceutically acceptable salt thereof for the manufacture of a medicament for the treatment of rhinitis.

In a yet further aspect, there is provided a method for the treatment of rhinitis which comprises administering to a patient in need thereof an effective amount of N-(2-bromophenyl)-N′-[((R)-1-(5-trifluoromethyl-2-pyridyl)pyrrolidin-3-yl)]urea or a pharmaceutically acceptable salt thereof.

Where used herein the term rhinitis is to be understood to include both allergic and non allergic rhinitis. Examples of non-allergic rhinitis include vasomotor rhinitis, irritant rhinitis, occupational rhinitis and NARES (non allergic rhinitis with eosinophils). In one embodiment N-(2-bromophenyl)-N′-[((R)-1-(5-trifluoromethyl-2-pyridyl)pyrrolidin-3-yl)]urea or a pharmaceutically acceptable salt thereof is used in the treatment of non allergic rhinitis.

N-(2-bromophenyl)-N′-[((R)-1-(5-trifluoromethyl-2-pyridyl)pyrrolidin-3-yl)]urea may be prepared by methods disclosed in patent application WO03/022809 (as Example 1) or by methods described herein.

N-(2-bromophenyl)-N′-[((R)-1-(5-trifluoromethyl-2-pyridyl)pyrrolidin-3-yl)]urea can form pharmaceutically acceptable salts. Suitable pharmaceutically acceptable salts are those used conventionally in the art and include those described in Berge, J. Pharm. Sci., 1977, 66, 1-19.

Suitable pharmaceutically acceptable salts include acid addition salts. Suitable pharmaceutically acceptable acid addition salts include salts with inorganic acids such, for example, as hydrochloric acid, hydrobromic acid, orthophosphoric acid or sulphuric acid, or with organic acids such, for example as methanesulphonic acid, toluenesulphonic acid, acetic acid, propionic acid, lactic acid, citric acid, fumaric acid, malic acid, succinic acid, salicylic acid, maleic acid, glycerophosphoric acid or acetylsalicylic acid.

In one embodiment N-(2-bromophenyl)-N′-[((R)-1-(5-trifluoromethyl-2-pyridyl)pyrrolidin-3-yl)]urea is in the form of a free base.

N-(2-bromophenyl)-N′-[((R)-1-(5-trifluoromethyl-2-pyridyl)pyrrolidin-3-yl)]urea may be prepared in crystalline or non-crystalline form. For example, N-(2-bromophenyl)-N′-[((R)-1-(5-trifluoromethyl-2-pyridyl)pyrrolidin-3-yl)]urea may be prepared in crystalline form by the procedure described in Scheme 1.

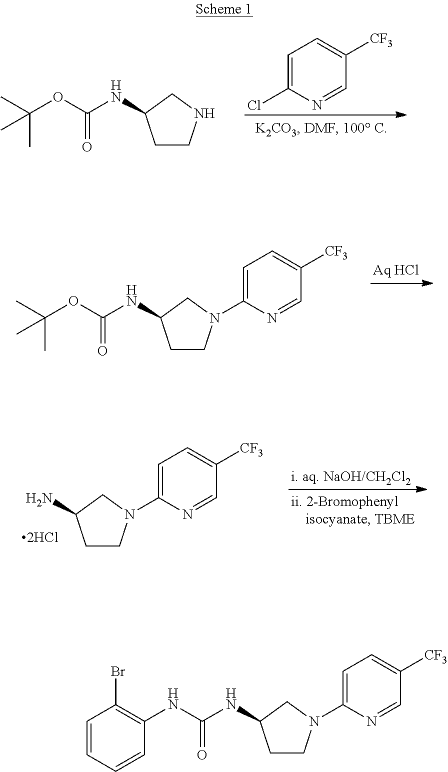

It will be appreciated that crystalline forms may be optionally hydrated or solvated. This invention includes in its scope stoichiometric hydrates as well as containing variable amounts of water. Suitable solvates include pharmaceutically acceptable solvates, such as hydrates. Solvates include stoichiometric solvates and non-stoichiometric solvates.

For use in this invention N-(2-bromophenyl)-N′-[((R)-1-(5-trifluoromethyl-2-pyridyl)pyrrolidin-3-yl)]urea or a pharmaceutically acceptable salt thereof may be formulated with one or more pharmaceutically acceptable excipients to provide a pharmaceutical composition. The pharmaceutical composition will be designed to suit the particular mode of administration.

In a further aspect of the present invention, there is provided an aqueous pharmaceutical composition comprising N-(2-bromophenyl)-N′-[((R)-1-(5-trifluoromethyl-2-pyridyl)pyrrolidin-3-yl)]urea or a pharmaceutically acceptable salt thereof, in particular a composition adapted for intranasal administration.

The aqueous pharmaceutical composition of the invention may be in the form of an aqueous suspension or an aqueous solution. In one embodiment, the aqueous pharmaceutical composition of the invention is in the form of an aqueous suspension.

The aqueous component is preferably a high grade quality of water, in particular purified water.

For use in this invention, N-(2-bromophenyl)-N′-[((R)-1-(5-trifluoromethyl-2-pyridyl)pyrrolidin-3-yl)]urea or a pharmaceutically acceptable salt thereof would typically be in a particle-size-reduced form, which may be prepared by conventional techniques, for example, microfluidisation, micronisation and milling e.g. wet bead milling. Generally, the size-reduced (e.g. micronised) N-(2-bromophenyl)-N′-[((R)-1-(5-trifluoromethyl-2-pyridyl)pyrrolidin-3-yl)]urea or a pharmaceutically acceptable salt thereof can be defined by a D₅₀ value of about 0.1 to 10 microns such as about 0.5 to 10 microns, more particularly about 2 to 4 microns (for example as measured using laser diffraction).

The proportion of N-(2-bromophenyl)-N′-[((R)-1-(5-trifluoromethyl-2-pyridyl)pyrrolidin-3-yl)]urea or a pharmaceutically acceptable salt thereof will depend on the precise type of composition to be prepared, but will generally be within the range of from about 0.01 to 20% (w/w), based on the total weight of the composition. Generally, however for most types of preparations the proportion used will be within the range of from about 0.05 to 10% (w/w), such as about 0.1 to 5% (w/w).

The dose of N-(2-bromophenyl)-N′-[((R)-1-(5-trifluoromethyl-2-pyridyl)pyrrolidin-3-yl)]urea or a pharmaceutically acceptable salt thereof will vary in the usual way with the seriousness of the disease to be treated and other factors such as the weight of the sufferer. As a general guide suitable unit doses may be about between 0.005 and 1 mg for example between 0.005 and 0.5 mg per dose. Such unit doses may be administered once a day, or more than once a day, for example two or three times a day. Such therapy may extend for a number of weeks or months.

Optionally a further active ingredient may be incorporated into the aqueous pharmaceutical composition, particularly one used in the treatment of rhinitis and suitable for intra-nasal administration such as an anti-histamine or a corticosteroid.

For use in combination, suitable examples of anti-histamines include azelastine, olopatadine, bepotastine or a compound selected from:
N-[2-((2R)-2-{[4-[(4-chlorophenyl)methyl]-1-oxo-2(1H)-phthalazinyl]methyl}-1-pyrrolidinyl)ethyl]-4-(methyloxy)butanamide (as disclosed in patent application WO2008/74803);
4-[(4-chlorophenyl)methyl]-2-({(2R)-1-[4-(4-{[3-(hexahydro-1H-azepin-1-yl)propyl]oxy}phenyl)butyl]-2-pyrrolidinyl}methyl)-1(2H)-phthalazinone (as disclosed in patent application WO2007/122156); or
N-(4-{4-[(6-butyl-8-quinolinyl)oxy]-1-piperidinyl}butyl)ethanesulfonamide (as disclosed in patent application PCT/EP2008/060622, published as WO2009/021965).

For use in combination, suitable examples of corticosteroids include fluticasone propionate (which is marketed as an intranasal formulation under the tradename Flixonase®), beclomethasone dipropionate (which is marketed as an intranasal formulation under the tradename Beconase®) or fluticasone furoate (which is marketed under the tradename Veramyst®). In one embodiment the present invention provides for an aqueous pharmaceutical composition comprising N-(2-bromophenyl)-N′-[((R)-1-(5-trifluoromethyl-2-pyridyl)pyrrolidin-3-yl)]urea or a pharmaceutically acceptable salt thereof and fluticasone furoate.

When present the proportion of the further active ingredient will generally be in the range from about 0.05 to 10% (w/w), such as about 0.1 to 5% (w/w).

Aqueous pharmaceutical compositions of the invention, such as intranasal compositions, may include one or more pharmaceutically acceptable excipients selected from the group consisting of suspending agents, thickening agents, preservatives, wetting agents and isotonicity adjusting agents.

Accordingly in one embodiment, there is provided an aqueous pharmaceutical composition comprising N-(2-bromophenyl)-N′-[((R)-1-(5-trifluoromethyl-2-pyridyl)pyrrolidin-3-yl)]urea or a pharmaceutically acceptable salt thereof and a suspending agent.

The suspending agent, if included, will typically be present in an amount of between about 0.1 and 5% (w/w), such as between about 1.5% and 2.5% (w/w), based on the total weight of the composition. Examples of suspending agents include Avicel®, carboxymethylcellulose, veegum, tragacanth, bentonite, methylcellulose and polyethylene glycols, e.g. microcrystalline cellulose or carboxy methylcellulose sodium.

In one embodiment, there is provided an aqueous pharmaceutical composition comprising N-(2-bromophenyl)-N′-[((R)-1-(5-trifluoromethyl-2-pyridyl)pyrrolidin-3-yl)]urea or a pharmaceutically acceptable salt thereof and a preservative.

For stability purposes, that compositions of the invention may be protected from microbial or fungal contamination and growth by inclusion of a preservative. Examples of pharmaceutically acceptable anti-microbial agents or preservatives may include quaternary ammonium compounds (e.g. benzalkonium chloride, benzethonium chloride, cetrimide and cetylpyridinium chloride), mercurial agents (e.g. phenylmercuric nitrate, phenylmercuric acetate and thimerosal), alcoholic agents (e.g. chlorobutanol, phenylethyl alcohol and benzyl alcohol), antibacterial esters (e.g. esters of para-hydroxybenzoic acid), chelating agents such as disodium ethylenediaminetetraacetate (EDTA) and other anti-microbial agents such as chlorhexidine, chlorocresol, sorbic acid and its salts (such as potassium sorbate) and polymyxin. Examples of pharmaceutically acceptable anti-fungal agents or preservatives may include sodium benzoate. The preservative, if included, may be present in an amount of between about 0.001 and 1% (w/w), such as about 0.015% (w/w), based on the total weight of the composition.

In another embodiment, there is provided an aqueous pharmaceutical composition which is preservative free.

In one embodiment, there is provided an aqueous pharmaceutical composition comprising N-(2-bromophenyl)-N′-[((R)-1-(5-trifluoromethyl-2-pyridyl)pyrrolidin-3-yl)]urea or a pharmaceutically acceptable salt thereof and a wetting agent.

Compositions which contain a suspended medicament may include a pharmaceutically acceptable wetting agent which functions to wet the particles of medicament to facilitate dispersion thereof in the aqueous phase of the composition. Typically, the amount of wetting agent used will not cause foaming of the dispersion during mixing. Examples of wetting agents include fatty alcohols, esters and ethers, such as polyoxyethylene (20) sorbitan monooleate (Polysorbate 80). The wetting agent may be present in an amount of between about 0.001 and 1.0% (w/w), such as between about 0.001 and 0.05% (w/w), for example about 0.025% (w/w), based on the total weight of the composition.

In one embodiment, there is provided an aqueous pharmaceutical composition comprising N-(2-bromophenyl)-N′-[((R)-1-(5-trifluoromethyl-2-pyridyl)pyrrolidin-3-yl)]urea or a pharmaceutically acceptable salt thereof and an isotonicity adjusting agent.

An isotonicity adjusting agent may be included to achieve isotonicity with body fluids e.g. fluids of the nasal cavity, resulting in reduced levels of irritancy. Examples of isotonicity adjusting agents include sodium chloride, dextrose, xylitol and calcium chloride. An isotonicity adjusting agent may be included in an amount of between about 0.1 and 10% (w/w), such as about 5.0% (w/w), based on the total weight of the composition.

Further, the aqueous pharmaceutical compositions comprising N-(2-bromophenyl)-N′-[((R)-1-(5-trifluoromethyl-2-pyridyl)pyrrolidin-3-yl)]urea or a pharmaceutically acceptable salt thereof may be buffered by the addition of suitable buffering agents such as sodium citrate, citric acid, phosphates such as disodium phosphate (for example the dodecahydrate, heptahydrate, dihydrate and anhydrous forms) or sodium phosphate and mixtures thereof.

Compositions of the invention e.g. those suitable for intranasal administration may also optionally contain other excipients, such as antioxidants (for example sodium metabisulphite), taste-masking agents (such as menthol) and sweetening agents (for example dextrose, glycerol, saccharin and/or sorbitol).

In one embodiment there is provided an aqueous pharmaceutical composition which comprises:

(i) an aqueous suspension of N-(2-bromophenyl)-N′-[((R)-1-(5-trifluoromethyl-2-pyridyl)pyrrolidin-3-yl)]urea or a pharmaceutically acceptable salt thereof;
(ii) one or more suspending agents;
(iii) one or more preservatives;
(iv) one or more wetting agents; and
(v) one or more isotonicity adjusting agents.

Aqueous pharmaceutical compositions according to the invention can be prepared using standard procedures that are familiar to the person skilled in the art e.g. by admixture of the various components, suitably at ambient temperature and atmospheric pressure.

In one embodiment, the aqueous pharmaceutical compositions of the invention are suitable for intranasal administration.

Intranasal compositions comprising N-(2-bromophenyl)-N′-[((R)-1-(5-trifluoromethyl-2-pyridyl)pyrrolidin-3-yl)]urea or a pharmaceutically acceptable salt thereof may permit the compound(s) to be delivered to all areas of the nasal cavities (the target tissue) and further, may permit the compound(s) to remain in contact with the target tissue for longer periods of time. A suitable dosing regime for intranasal compositions would be for the patient to inhale slowly through the nose subsequent to the nasal cavity being cleared. During inhalation the composition would be administered to one nostril (for example, as a spray or drops) while the other is manually compressed. This procedure would then be repeated for the other nostril. Typically, one or two sprays per nostril would be administered by the above procedure up to two or three times each day, ideally once daily.

The compositions of the invention are provided in a suitable container. Aqueous pharmaceutical compositions which are non pressurized and adapted to be administered topically to the nasal cavity are of particular interest. Aqueous compositions may also be administered to the nose by nebulisation.

Accordingly, there is provided a container comprising an aqueous pharmaceutical composition of the invention suitable for delivering said composition to the nasal cavities.

Typically the composition of the present invention will be packaged into a suitable container being a fluid dispenser e.g. a multi-dose container with a nasal applicator, wherein the dose is capable of being metered by volume.

Such a fluid dispenser may typically have a dispensing nozzle or dispensing orifice through which a metered dose of the fluid composition is dispensed upon the application of a user-applied force to a pump mechanism of the fluid dispenser. Such fluid dispensers are generally provided with a reservoir of multiple metered doses of the fluid composition, the doses being dispensable upon sequential pump actuations. The dispensing nozzle or orifice may be configured for insertion into the nostrils of the user for spray dispensing of the fluid composition into the nasal cavity. A fluid dispenser of the aforementioned type is described and illustrated in WO05/044354 the entire content of which is hereby incorporated herein by reference. The dispenser has a housing which houses a fluid discharge device having a compression pump mounted on a container for containing a fluid composition. The housing has at least one finger-operable side lever which is movable inwardly with respect to the housing to cam the container upwardly in the housing to cause the pump to compress and pump a metered dose of the composition out of a pump stem through a nasal nozzle of the housing. In one embodiment, the fluid dispenser is of the general type illustrated in FIGS. 30-40 of WO05/044354.

The following examples illustrate the preparation of the aqueous pharmaceutical compositions and use thereof in accordance with this invention. The Examples are to be considered illustrating and not limiting the scope of the disclosure in any way.

### Characterisation of a crystalline form of N-(2-bromophenyl)-N′-[((R)-1-(5-trifluoromethyl-2-pyridyl)pyrrolidin-3-yl)]urea (Compound 1)

A crystalline form of Compound 1 made, for example, by the procedure described in Scheme 1 was characterised by X-ray powder diffraction (XRPD) and Differential Scanning calorimetry (DSC).

### X-Ray Powder Diffraction (XRPD)

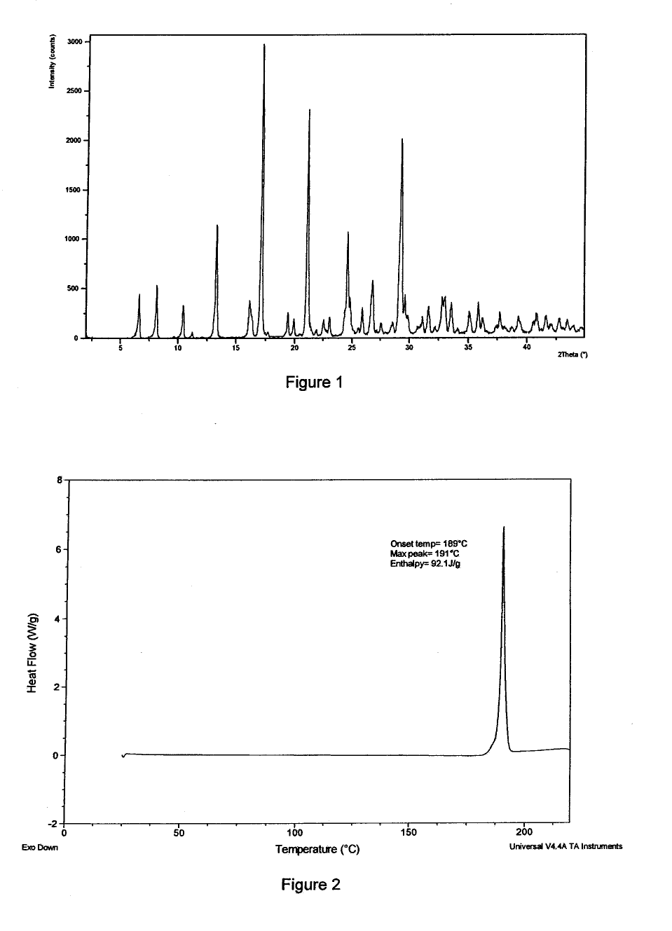

FIG. 1 shows the diffraction pattern for a crystalline form of Compound 1 which was recorded on a PANalytical X′Pert Pro powder diffractometer, model PW3040/60, serial number DY667 using a point detector. The acquisition conditions were: radiation: Cu Kα, generator tension: 40 kV, generator current: 50 mA, start angle: 2.0° 2θ, end angle: 45.0° 2θ, step size: 0.02° 2θ, time per step: 1.0 seconds. The sample was prepared by loading a sample cup using a front-filled approach, resulting in a thin layer of powder. Characteristic XRPD angles and d-spacings are recorded in Table 1. The margin of error is approximately ±0.1° 2θ for each of the peak assignments. Peak positions were measured using Highscore software.

TABLE 1

| 2θ/° | d-spacing/Å |
|---|---|
| 6.7 | 13.3 |
| 8.2 | 10.8 |
| 9.7 | 9.1 |
| 10.5 | 8.4 |
| 11.3 | 7.9 |
| 13.3 | 6.6 |
| 16.1 | 5.5 |
| 17.2 | 5.2 |
| 17.7 | 5.0 |
| 19.5 | 4.6 |
| 20.0 | 4.4 |
| 21.2 | 4.2 |
| 22.6 | 3.9 |
| 23.1 | 3.8 |
| 24.7 | 3.6 |
| 24.9 | 3.6 |
| 25.9 | 3.4 |
| 26.9 | 3.3 |
| 29.3 | 3.0 |
| 29.7 | 3.0 |
| 31.7 | 2.8 |

### Differential Scanning Calorimetry (DSC)

FIG. 2 shows the DSC thermogram obtained using a TA Q100 calorimeter, serial number 0100-0332. The sample was weighed into an aluminium pan, a pan lid placed on top and lightly crimped without sealing the pan. The experiment was conducted using a heating rate of 10° C. min⁻¹.

### EXAMPLE 1

### Preparation of an aqueous pharmaceutical composition comprising N-(2-Bromophenyl)-N′-[((R)-1-(5-trifluoromethyl-2-pyridyl)pyrrolidin-3-yl)]urea (Compound 1)

The aqueous pharmaceutical compositions of the invention may be prepared according to the following general method.

The isotonicity adjusting agent(s) is charged into a suitable mixing vessel containing purified water and dissolved with stirring. Preservative(s) is pre-dissolved in purified water in a separate vessel, optionally with heating, for example to 50-60° C. depending on the preservative chosen, to aid dissolution, and then added to the isotonicity adjusting agent(s) with continuous stirring. The suspending agent(s) is then charged into the mixing vessel and dispersed throughout the solution. The resulting suspending vehicle is allowed to hydrate for an appropriate period of time to ensure cross-linkage and gelation, which may take 60 minutes or longer.

In a separate mixing vessel, the wetting agent(s) is mixed with purified water which optionally may be heated, for example to about 50-60° C. as appropriate depending on the wetting agent(s) chosen, and stirred to dissolve. A slurry of Compound 1 or a pharmaceutically acceptable salt thereof (alone or in combination with a further active ingredient) is then prepared by adding the resultant wetting agent(s) solution to the active compound(s), which may be particle size reduced for example micronised, and mixed prior to homogenising/refining. Additionally, in a separate mixing vessel, additional preservative(s), if needed, may be diluted with purified water and stirred to mix.

Following the dispersion and gelation the slurry of active compound(s) is added to the mixing vessel containing the suspending agent and dispersed with stirring. Following the addition of the slurry of active compound(s), any additional preservative may be added to the bulk suspension and dispersed with continuous stirring. Finally, the suspension is made to its final mass by adding water and stirred.

### EXAMPLE 2

### Characterisation, quantification and dose response effects of pre-treatment with N-(2-Bromophenyl)-N′-[((R)-1-(5-trifluoromethyl-2-pyridyl)pyrrolidin-3-yl)]urea (Compound 1) on contralateral nasal secretory response following an ipsilateral nasal challenge in the guinea pig

### General Experimental Design

Dunkin Hartley guinea pigs (ca. 180-200 g) were intranasally sensitised into both nostrils twice daily for 5 days with 25 μl of 10 μg ovalbimun plus 10 mg aluminium hydroxide in saline. This was followed up with one week of intranasal challenge in both nostrils with ovalbumin 25 μl of a 5 mg/ml saline solution per nostril (weekends eliminated). After this period animals were maintained on this regime until the day of the study when they did not receive an ovalbumin intranasal challenge.

Six animals per group were studied and a repeat of each study conducted to provide up to 12 animals per group in the final data set. Animals were pretreated with Compound 1 (intranasal administration 25 μl per nostril of a suspension or oral administration 1 ml of a solution in 50% deionised water 5% DMSO and 45% PEG 200). At a set time point the animals were anaethetised with urethane (1.5 g/kg) i.p. and scanned with magnetic resonance imaging (MRI) to obtain baseline nasal images. Each animal was then removed from the scanner and received an ipsilateral nasal challenge of capsaicin (50 μl of a solution 0.3 mM 5% DMSO, 5% Tween 80, 90% Phosphate buffered saline)) or 10% hypertonic saline solution (50 μl) (administered by pipette. The animal was rescanned at 10 minutes post-challenge to measure the contralateral fluid volumes.

The fluid measurement was made using standard T2 weighted spin echo MRI sequences on a 2 Tesla Bruker Medspec MRI scanner. The raw data was collected using Paravision 3.0.2 (Bruker) software. Fluid measurements were made using grey level thresholding technique using Mayo Clinic software Analyze 7.0. Statistical analysis was carried out using the Statistica 6.0 software by StatSoft

### Model Validation

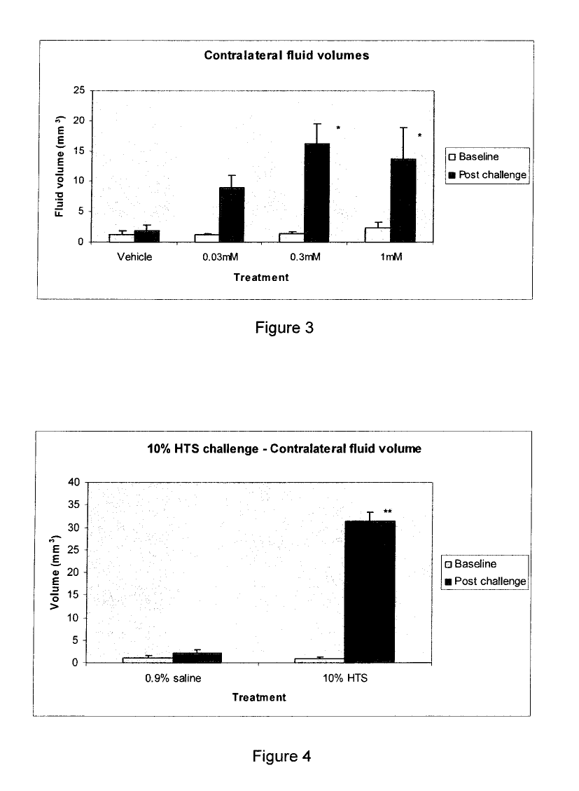

The effects of the capsaicin and hypertonic saline (HTS) challenge on contralateral fluid secretion are illustrated in FIGS. 3 and 4 respectively.

FIG. 3: Shows the effects of capsaicin ipsilateral challenge on contralateral fluid secretion
Baseline measurements=pre capsaicin challenge, 10 mins data point=10 min post 0.3 mM capsaicin ipsilateral challenge.

Statistical significance (p*<0.05; compared with capsaicin vehicle challenge group. n=6) was achieved at both 0.3 mM and 1 mM capsaicin. The optimal capsaicin challenge dose was then set at 0.3 mM for all subsequent studies.

FIG. 4: Shows the effects of hypertonic saline (HTS) challenge on contralateral fluid secretion
Baseline measurements=pre capsaicin challenge, Post treatment=10 min post hypertonic saline ipsilateral challenge. A substantial and very consistent contralateral fluid response was seen in response to 50 μl 10% hypertonic saline challenge (p<0.0001) ANOVA with Dunnett's followup test.

### Effects of administration of N-(2-Bromophenyl)-N′-[((R)-1-(5-trifluoromethyl-2-pyridyl)pyrrolidin-3-yl)]urea

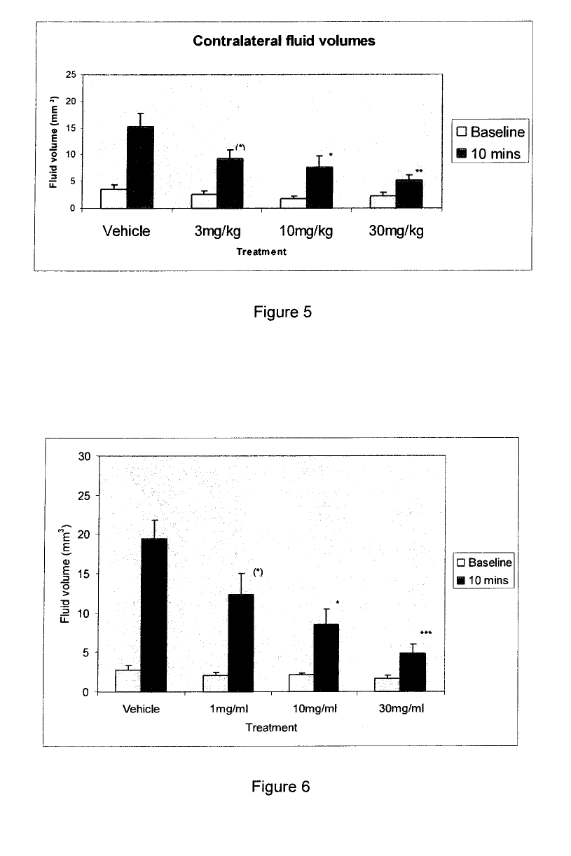

Oral administration of N-(2-bromophenyl)-N′-[((R)-1-(5-trifluoromethyl-2-pyridyl)pyrrolidin-3-yl)]urea 1 hour prior to capsaicin challenge gave significant inhibition of contralateral fluid secretion; ca. 50% reduction at 10 mg/kg (p<0.05) and ca. 70% at 30 mg/kg (p<0.001) as illustrated in FIG. 5.

FIG. 5: Shows the effects of 1 hour pre-treatment of oral non micronised Compound 1 on capsaicin provoked nasal secrection.
Baseline measurements=pre capsaicin challenge, 10 mins data point=10 min post 0.3 mM capsaicin ipsilateral challenge.
(*) p=0.06; p*<0.05; **p<0.001 compared with vehicle pre-treatment group ANOVA with Dunnett's followup test

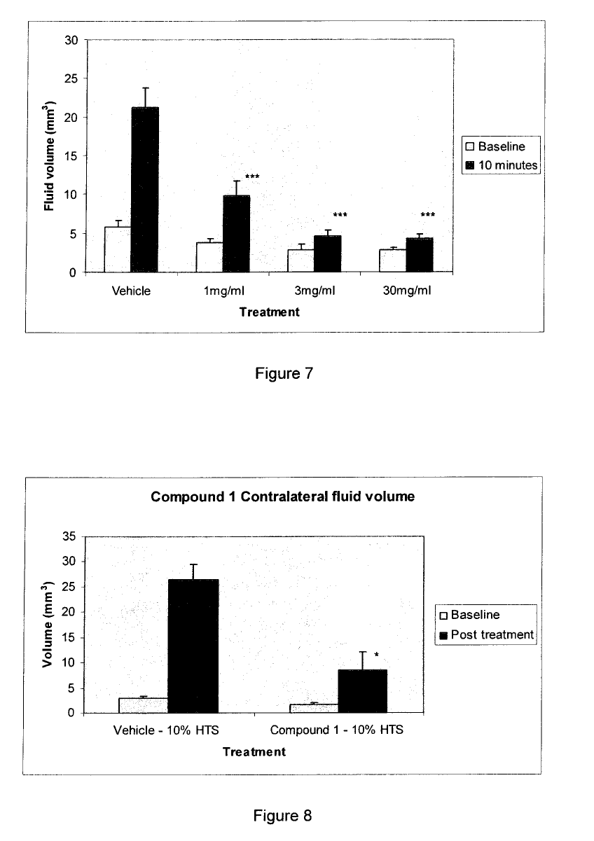

The effects of topical pre-treatment with non-micronised and micronised N-(2-bromophenyl)-N′-[((R)-1-(5-trifluoromethyl-2-pyridyl)pyrrolidin-3-yl)]urea are illustrated in FIG. 6 and FIG. 7 respectively.

FIG. 6: Shows the effect of 1 hour pre-treatment of topical non-micronised Compound 1 on capsaicin provoked nasal secrection
Baseline measurements=pre capsaicin challenge, 10 mins data point=10 min post 0.3 mM capsaicin ipsilateral challenge.
(*) p<0.07; p*<0.005; **p<0.0001 compared with vehicle pre-treatment group ANOVA with Dunnett's followup test

With non-micronised material >50% significant inhibition was observed in the 10 mg/ml (p<0.005) and ca. 75% significant inhibition observed in the 30 mg/ml pre-treated animals.

FIG. 7: Shows the effect of 1 hour pre-treatment of topical micronised Compound 1 on capsaicin provoked nasal secrection
Baseline measurements=pre capsaicin challenge, 10 mins data point=10 min post 0.3 mM capsaicin ipsilateral challenge.
***p<0.0005 compared with vehicle pre-treatment group, ANOVA with Dunnett's followup test

Comparison of FIGS. 5 and 6 demonstrates an improvement in the inhibition of nasal secretion when N-(2-bromophenyl)-N′-[((R)-1-(5-trifluoromethyl-2-pyridyl)pyrrolidin-3-yl)]urea was administered topically. It is clear from FIGS. 6 and 7 that particle size reduction by micronisation gave significant inhibition of the capsaicin response at all the doses evaluated.

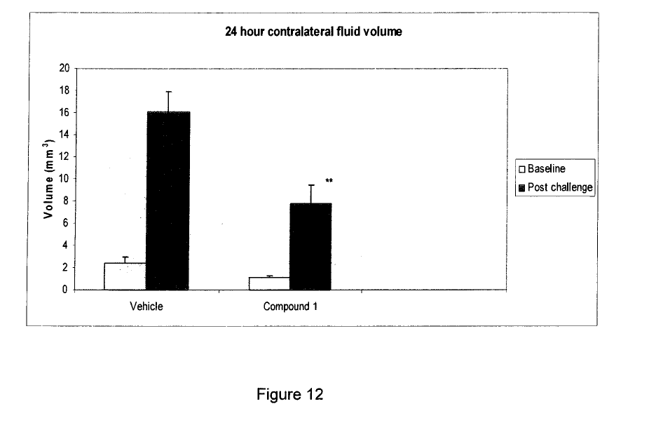

The duration of effect of pre-treatment with micronised N-(2-bromophenyl)-N′-[((R)-1-(5-trifluoromethyl-2-pyridyl)pyrrolidin-3-yl)]urea are illustrated in FIG. 11 and FIG. 12 respectively. A significant inhibition, 62% inhibition after 12 hours pre-treatment and 52% inhibition after 24 hours pre-treatment, was observed in contralateral response when the animals were given a capsaicin challenge 12 and 24 hours after pre-treatment with compound 1.

FIG. 11: Shows the effect of 12 hour pre-treatment of topical micronised Compound 1 on capsaicin provoked nasal secrection
Baseline measurements=pre capsaicin challenge, 10 mins data point=10 min post 0.3 mM capsaicin ipsilateral challenge.
**p<0.0001 compared with vehicle pre-treatment group, ANOVA with Dunnett's followup test

FIG. 12: Shows the effect of 24 hour pre-treatment of topical micronised Compound 1 on capsaicin provoked nasal secrection
Baseline measurements=pre capsaicin challenge, 10 mins data point=10 min post 0.3 mM capsaicin ipsilateral challenge.
**p<0.0001 compared with vehicle pre-treatment group, ANOVA with Dunnett's followup test

The ability of N-(2-bromophenyl)-N′-[((R)-1-(5-trifluoromethyl-2-pyridyl)pyrrolidin-3-yl)]urea to inhibit the response to alternative challenges was evaluated using the hypertonic saline challenge, pre-treatment with 1 mg/ml micronised N-(2-bromophenyl)-N′-[((R)-1-(5-trifluoromethyl-2-pyridyl)pyrrolidin-3-yl)]urea resulted in significant inhibition (68%) of the hypertonic saline response (see FIG. 8).

FIG. 8: Shows the effects of 1 hour pre-treatment of topical micronised Compound 1 on hypertonic saline provoked nasal secretion
Baseline measurements=pre capsaicin challenge, Post treatment=10 min post hypertonic saline ipsilateral challenge.
p<0.005 compared with vehicle pre-treatment group, ANOVA with Dunnett's followup test

### EXAMPLE 3

### Electrophysiological Characterisation of the Effects of N-(2-bromophenyl)-N′-[((R)-1-(5-trifluoromethyl-2-pyridyl)pyrrolidin-3-yl)]urea (Compound 1) on Capsaicin Induced Currents recorded from Nasally Innervated Guinea-Pig Trigeminal Ganglia Cell Bodies

### Method

Nasal afferent neurons were retrogradely labelled by using DiIC13(3) solution (2% in DMSO, sonicated to ensure solubility) (Dil, Invitrogen), A guinea pig was dosed with atropine (1 mg/kg i.p.) 5 minutes prior to anaesthesia with isoflurane. Once unconscious 25 μl of the Dil solution was placed into the right nostril and the guinea pig allowed to recover. After 24 hours the procedure was repeated and the left nostril dosed.

After a period of 2 weeks the guinea pig was killed via CO₂ asphyxiation and the trigeminal ganglia rapidly dissected. An area of approximately 3 mm×2 mm which contained neurons that innervated the nose was isolated (J. Allergy Clin. Immunol. 2005, 116, 1282), cut into smaller pieces and incubated in enzyme buffer (8 mg papain in 4 ml L-15) at 37° C. for 40 min. The papain solution was removed and the tissue washed three times with 5-5 media (5-5 Media was prepared by the addition of 5 ml Fetal Bovine Serum, 5 ml Horse Serum, 1 ml 5000 Units Penicillin/5000 μm Streptomycin, 1 ml 30% Glucose and 0.33 ml of 1× stock Insulin Transferrin Sodium Selenite to 90 ml Minimum Essential Media with Earle's salts and Glutamax). The neurons were dissociated by trituration with 3 fire-polished glass Pastuer pipettes of decreasing tip size, then the resulting cell suspension filtered and centrifuged (1000 g for 4 min). The pellet was resuspended in 300 μl of 5-5 media. 25 μl of the cell suspension was plated onto poly-D-lysine and laminin coated 12 mm glass coverslips and placed in an incubator at 37° C. After 2 hours the coverslips were flooded with 5-5 media and used within 48 hours.

Conventional whole cell patch clamp techniques were employed under voltage clamp (holding potential of −70 mV) at room temperature. Drug solutions were applied to the cell using a Rapid Solution Changer (Biologic). Cell bodies that had innervated the nose were identified using a fluorescence based microscope.

All data was analysed using pClamp version 10.2.0.9 and 10.2.0.10 (Molecular Devices Inc). Subsequently this data was graphically represented using Graphpad Prism (Version 5.0) and a non linear regression fitted using the built in equation log(agonist) vs normalised response−variable slope) (also known as the Hill equation) to generate an IC₅₀ value and Hill coefficient.

### Effects of N-(2-bromophenyl)-N′-[((R)-1-(5-trifluoromethyl-2-pyridyl)pyrrolidin-3-yl)]urea

Application of 1 μM capsaicin to small diameter (<30 μm) fluorescent neurons voltage clamped at −70 mV produced an inward current in 56% of the cells tested. Over a 60 second period this current exhibited little or no macroscopic desensitisation.

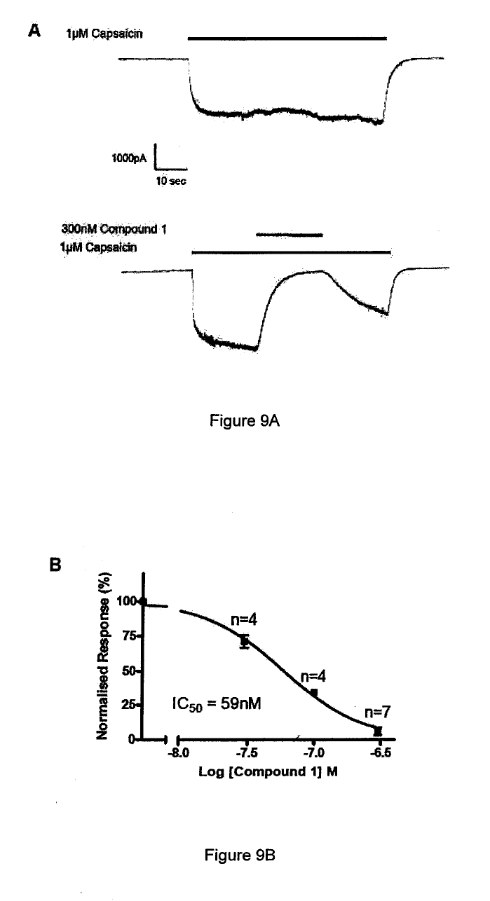

Compound 1 produced a concentration related inhibition of the capsaicin response. This inhibition showed rapid reversal upon switching from capsaicin and Compound 1 to capsaicin only. (FIG. 9 Part A)

Pooled data from a number of similar experiments (n>4) were fitted with the Hill equation and gave an IC₅₀ of 59 nM for the Compound 1 (FIG. 9 Part B).

### EXAMPLE 4

### Characterisation, quantification and dose response effects of pre-treatment with N-(2-Bromophenyl)-N′-[((R)-1-(5-trifluoromethyl-2-pyridyl)pyrrolidin-3-yl)]urea (Compound 1) on contralateral nasal tissue volume following an ipsilateral nasal challenge in the guinea pig

Using an similar general experimental procedure as described for Example 2 it was also possible to evaluate contralateral nasal tissue volume changes following an ipsilateral capsaicin challenge.

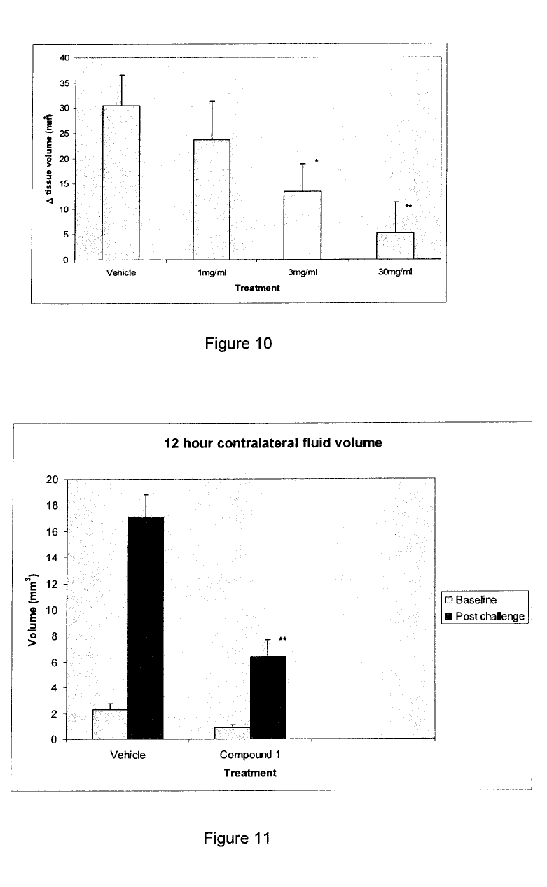

Following pre-treatment with the vehicle a capsaicin challenge produced a substantial contralateral tissue swelling of approximately 30 mm². Pre-treatment with Compound 1 exhibited a reduced degree of swelling in a dose dependent manner. The results are illustrated in FIG. 10, at 3 mg/ml and 30 mg/ml a reduction of 56% (p<0.05) and 83% (p<0.01) was observed when compared to the vehicle control.

FIG. 10: Shows the effect of 1 hour pre-treatment of topical micronised Compound 1 on capsaicin provoked nasal tissue response
Baseline measurements=pre capsaicin challenge, 10 mins data point=10 min post 0.3 mM capsaicin ipsilateral challenge.
(*) p<0.05; **p<0.01 compared with vehicle pre-treatment group, n=10/12 per group. ANOVA with Dunnett's followup test

All publications, including but not limited to patents and patent applications, cited in this specification are herein incorporated by reference as if each individual publication were specifically and individually indicated to be incorporated by reference herein as though fully set forth.

What is claimed is:

1-3. (canceled)

4. A method for the treatment of rhinitis which comprises administering to a patient in need thereof an effective amount of N-(2-bromophenyl)-N′-[((R)-1-(5-trifluoromethyl-2-pyridyl)pyrrolidin-3-yl)]urea or a pharmaceutically acceptable salt thereof.

5. An aqueous pharmaceutical composition comprising N-(2-bromophenyl)-N′-[((R)-1-(5-trifluoromethyl-2-pyridyl)pyrrolidin-3-yl)]urea or a pharmaceutically acceptable salt thereof.

6. An aqueous pharmaceutical composition according to claim 5 in which N-(2-bromophenyl)-N′-[((R)-1-(5-trifluoromethyl-2-pyridyl)pyrrolidin-3-yl)]urea is in the form of a free base.

7. An aqueous pharmaceutical composition according to claim 5 in which N-(2-bromophenyl)-N′-[((R)-1-(5-trifluoromethyl-2-pyridyl)pyrrolidin-3-yl)]urea or a pharmaceutically acceptable salt thereof has a particle size D₅₀ value of about 0.5 to 10 microns.

8. An aqueous pharmaceutical composition according to claim 5 which is in the form of an aqueous suspension.

9. An aqueous pharmaceutical composition according to claim 5 further comprising one or more pharmaceutically acceptable excipients selected from the group consisting of suspending agents, thickening agents, preservatives, wetting agents and isotonicity adjusting agents.

10. An aqueous pharmaceutical composition according to claim 9 which comprises:
(i) an aqueous suspension of N-(2-bromophenyl)-N′-[((R)-1-(5-trifluoromethyl-2-pyridyl)pyrrolidin-3-yl)]urea or a pharmaceutically acceptable salt thereof;
(ii) one or more suspending agents;
(iii) one or more preservatives;
(iv) one or more wetting agents; and
(v) one or more isotonicity adjusting agents.

11. An aqueous pharmaceutical composition according to claim 5 comprising a further active ingredient which is an anti-histamine or a corticosteroid.

12. A container comprising an aqueous pharmaceutical composition according to claim 5 suitable for delivering said composition to the nasal cavities.
